(19) United States
(12) Patent Application Publication
(10) Pub. No.: US 2011/0044950 A1
(43) Pub. Date: Feb. 24, 2011

(54) Infusion of a Mixture of Autologous Bone Marrow-Derived Mononuclear Cells and Autologous or Allogeneic Bone Marrow-Derived Mesenchymal Stem Cells for Treating Myocardial and/or Cardiovascular Disorders

(76) Inventors: Jose J. Minguell, Covington, LA (US); Gabriel P. Lasala, Covington, LA (US)

Correspondence Address: Adams and Reese LLP, 1221 McKinney Street, Suite 4400, Houston, TX, 77010, US

(21) Appl. No.: 12/939,041

(22) Filed: Nov. 3, 2010

Related U.S. Application Data
(63) Continuation-in-part of application No. 12/456,318, filed on Jun. 15, 2009.
Application No. 12/456,318 is a continuation of application No. 11/500,317, filed on Aug. 8, 2006, now abandoned.

(51) Int. Cl. A61K 35/12; A61K 35/32

(52) U.S. Cl. 424/93.3

(57) ABSTRACT

The present invention is a method for improving cardiac and/or cardiovascular functions in living subjects after the occurrence of a myocardial and/or cardiovascular disorder, involving tissue damage and/or an ischemic event. The method is a combination stem cell therapy involving a mixture of bone marrow-derived mesenchymal stem cells and bone marrow-derived mononuclear cells surgically implanted by using either a direct or catheter-mediated injection into damaged tissue. Studies have shown that the implant improves blood perfusion in an ischemic tissue and thus contributes to the recovery of cardiac and/or cardiovascular function as assessed by methods of choice, including magnetic resonance imaging (“MRI”), echocardiography, angiography and 99mTc-TF perfusion scintigraphy.

### CROSS-REFERENCES TO RELATED APPLICATIONS

The present application is a continuation-in-part of U.S. patent application Ser. No. 12/456,318, filed Jun. 15, 2009, still pending, which was a continuation of U.S. patent application Ser. No. 11/500,317, filed Aug. 8, 2006, now abandoned. Applicant hereby claims the benefit of and incorporates by reference U.S. patent application Ser. Nos. 12/456,318 and 11/500,317.

### FIELD OF THE INVENTION

The present invention relates generally to methods for treating myocardial and/or cardiovascular disorders, and more specifically, to methods for the regeneration and revascularization of the ischemic tissue.

### BACKGROUND OF THE INVENTION

Myocardial and/or cardiovascular disorders (also referred to herein as “CD”) is a widespread and important cause of morbidity in the United States and mortality amongst adults. Due to scar- and ischemia-related events, clinical manifestations are enormous and heterogeneous. In the case of myocardial dysfunction, the damaged left ventricle undergoes progressive “remodeling” and chamber dilation, with myocyte slippage and fibroblast proliferation. These events reflect an apparent lack of effective intrinsic mechanisms for myocardial repair and regeneration. Unless significant (and still unknown) modifications are introduced in the area proximate to the ischemic damage to force proliferation of resident cells (Beltrami, 2001), all restorative therapies for CD must consider the use of an exogenous source of cardiomyocyte repair progenitors.

Deciding the source and nature of cells to utilize for treatment has been a fertile area of scientific studies. According to preclinical studies, the choice has ranged from resident-differentiated but quiescent cardiomyocytes to stem cells or cardiomyocyte progenitors (Warejcka, 1996; Wang, 2000; Siminiak, 2003). Because a cardiac and/or a cardiovascular monopotential stem cell has not yet been identified, the clinical options are narrowed to the use of a multipotential stem cell exhibiting a potential to differentiate into the cardiomyocyte and/or cardiovascular lineage. From this point of view, bone marrow-located stem cells display the required biological properties for a cell therapy approach to treat patients with myocardial infarction (also referred to herein as “MI”) (Wulf, 2001; Wagers, 2002; Herzog, 2003). Using animal models, a near-normalization of ventricular function after treatment of acute infarcted myocardium with locally-injected bone marrow-derived precursor cells has been reported (Jackson, 2001; Orlic, 2001, for a recent review, see Husnain, 2005). However, it was not clear from the models whether the beneficial effect produced by the graft was elicited by hematopoietic stem cells, precursors for cardiomyocytes and/or endothelial cells, stem cell plasticity or just contamination with other marrow cells (Wagers, 2002). On the other hand, the transplantation of unfractionated sheep bone marrow into chronically infarcted myocardium did not result in any beneficial effect (Bel, 2003).

Several studies have utilized mesenchymal stem cells (also referred to herein as “MSCs” or “BM-MSCs”) as a cell archetype for regenerative purposes after CD. hi vitro studies have shown that MSCs have the potential to differentiate into spontaneous beating myotube-like structures, which express natriuretic peptides, myosin, desmin, and actinin, and exhibit sinus node-like and ventricular cell-like action potentials (Makino, 1999; Bittira, 2002). In vivo studies have shown that when MSCs are implanted into myocardium they undergo a milieu-dependent (microenvironment) cardiomyogenic differentiation and develop into myofibers containing striated sarcomeric myosin heavy chain and cell-to-cell junctions (Wang, 2000; Barbash, 2003). The xenogeneic or syngeneic transplantation of MSCs have shown that infused cells were signaled and recruited to the normal and/or injured heart (Allers, 2004; Bittira, 2002), where they undergo differentiation and participate in the pathophysiology of post-infarct remodeling, angiogenesis and maturation of the scar (Bittira, 2003; Pittenger, 2005; Minguell, 2006). Furthermore, recent pig studies have shown that allogenic MSC infusion improves left ventricular function following myocardial infarction with no detectable immune response or other toxicity (Min, 2002; Shake, 2002).

The results of experimental studies showing that the implant of bone marrow-derived progenitor cells improves heart function after CD have prompted several groups to test this notion in people. In the last three years, various clinical studies have assessed the effect of transplantation of autologous bone marrow in myocardial regeneration after acute myocardial infarction. In all of these studies, the source of “repairing” cells has been the bone marrow mononuclear cell fraction (also referred to herein as “BM-MNCs”), which contains B, T and NK lymphocytes, early myeloid cells, endothelial progenitors and a very low number of hematopoietic and/or mesenchymal stem cells. In these studies, bone marrow was aspirated (40-250 mL) from patients, the BM-MNCs then prepared and the resulting cells (10 ⁶ to 10 ⁷) implanted into the infarcted ischemic myocardium by using either a direct or a catheter-mediated injection. Results have shown that the autologous implantation procedure is safe, feasible and effective under clinical conditions (Assmus, 2002; Perin, 2003; Sekiya, 2002; Stamm, 2003; Strauer, 2002; Tse, 2003). In all cases, the observed therapeutic effect was attributed to bone marrow progenitors, associated to neovascularization (new blood vessels formation, angiogenesis; Rafii, 2003), thus improving perfusion of the ischemic tissue.

Currently, methods exist utilizing cell-based therapies for treating ischemia. In one method known in the art, both the BM-MSCs and the so-called endothelial generating cells are purified, expanded, and enriched. Laughlin et al., United States Patent Application Publication no. US 2004/0258670, application Ser. No. 10/730,549, filed Dec. 5, 2003, is hereby incorporated by reference.

BM-MSCs make up only a minute percentage of the total composition of the bone marrow aspirate, and, thus, must be expanded in order to obtain a therapeutically effective amount for treating the patient's condition. In the current art, the endothelial generating cells present in the bone marrow mononuclear cell fraction are also enriched and then expanded. However, enrichment and expansion of the endothelial generating cells creates a biological and safety risk, due to contamination and other factors, not to mention the extensive time and labor required to prepare the cells for infusion.

Thus, there is an unmet need in the art for a method of treating CD that is both safe and effective, while also reducing the time needed to create the treatment mixture.

Based on pre-clinical and clinical studies, the rationale of the present clinical study is the following: every clinical attempt for tissue regeneration after a CD might consider the implant of progenitor cells, with the potential to differentiate and mature into functional cells, thus contributing to the recovery of local contractility. However, a comprehensive therapy should also consider the revascularization of the ischemic tissue by the implant of endothelial progenitor cells (Minguell, 2010; Lasala, 2010; Lasala (in press) 2010).

### BRIEF SUMMARY OF INVENTION

The combined infusion of autologous or allogeneic purified and expanded bone marrow-derived mesenchymal stem cells (a source of cardiomyocyte and vascular progenitors) and autologous bone marrow mononuclear cells (a primary source of endothelial progenitors) represents an effective and enduring myocardial and cardiovascular replacement therapy. The above presupposes that the pair of implanted progenitors will express their respective biological programs after interacting with proper microenvironment locus of the receptor tissue (Minguell, 2001; Wagers, 2002; Rafii, 2003).

### DETAILED DESCRIPTION OF THE INVENTION

Results of experimental and clinical studies have shown that implantation of autologous bone marrow derived mononuclear cells (“BM-MNC”) induces neovascularisation, but not a robust improvement in tissue function, after MI and/or a cardiovascular disorder. In an embodiment of the present invention, we propose that the above therapy in conjunction with one that provides a source of cardiomyocytes and/or endothelial repair cells will represent a substantial promise as a cellular agent for cardiovascular therapy.

As a source of cardiomyocyte and/or endothelial repair progenitors and based on in vitro, ex vivo and in vivo studies, the present invention utilizes autologous or allogeneic ex vivo-expanded bone marrow-derived mesenchymal stem cells. Encouraging preliminary efficacy data in large animal models of myocardial infarction (Minguell, 2006) and accumulating safety data from human studies of MSCs in non-cardiovascular applications is encouraging.

In one example though non-limiting embodiment, the intracoronary injection (implant via catheter or direct injection) of a mixture of autologous or allogeneic BM-MSCs and autologous BM-MNCs represents an effective and enduring myocardial and/or cardiovascular replacement therapy. MSCs are not immunologically rejective, and, thus, do not need to come from the patient's bone marrow, but can instead come from a suitable human donor.

In an example embodiment, primary bone marrow aspirations from the iliac crest will be performed in the patient or a suitable donor twenty-five±five days before the patient is to receive the cell infusion. During the time between aspiration and infusion, the BM-MSCs will be prepared through ex vivo expansion and purification, until a therapeutically effective amount is obtained.

In another example though non-limiting embodiment, a secondary bone marrow aspiration will be performed in patients twenty-five±five days after the primary aspiration from the iliac crest. This aspiration is used for preparation of BM-MNCs and is performed, in an example embodiment, within five hours of the cell infusion to the patient. This example embodiment does not require purification or expansion of the endothelial progenitor cells, but instead, the content of endothelial progenitor cells biologically present in the bone marrow mononuclear cell fraction is used. For cell infusion, aliquots of autologous or allogeneic expanded BM-MSCs and autologous BM-MNCs are mixed together for a final solution of infusion medium.

In analyzing the bone marrow mononuclear cell fraction, to ensure a therapeutically effective amount of BM-MNCs was obtained in the second aspiration, a method known in the art is used to count the number of endothelial progenitor cells present in the BM-MNC fraction. Endothelial progenitor cells have a unique ligand configuration in the cell membrane which allows the preparer to easily and quickly ascertain the number of endothelial progenitor cells present in the aspiration.

For a better understanding of the above-described procedures and schedule, refer to Table 1 below.

TABLE 1
DIAGRAM OF PROCEDURES AND
SCHEDULE FOR PREPARATION OF MSCs and MNCs

| Days to | | Type of sample | Type of test to be |
|---|---|---|---|
| infusion | Step | to be taken | performed |
| −25 | 1^{st} Bone marrow aspirate | cell suspension | differential cell count; |
| | | | microbiological |
| −25 | Separation of the Mononuclear | cell suspension | differential cell count |
| | cell fraction | | |
| −20 | Passage #0 (Primary BM-MSC | growth medium | cell number, viability, |
| | culture) | & cell | microbiological |
| | | suspension | |
| −16 | Passage #1 | cell suspension | cell number, viability |
| −12 | Passage #2 | cell suspension | cell number, viability |
| −8 | Passage #3 | cell suspension | cell number, viability |
| −4 | Passage #4 (Expanded MSC) | growth medium | cell number, viability, |
| | | & cell | microbiological, |
| | | suspension | mycoplasma |
| 0 | Final preparation of BM-MSC | BM-MSC | cell number, viability, |
| | | suspension | immunotypification, |
| | | | differentiation potential |
| | | | microbiological, |
| | | | mycoplasma, Gram stain |
| 0 | 2^{nd} Bone marrow aspirate for | BM-MNC | cell number, viability, |
| | preparation of MNC cells | suspension | immunotypification, |
| | | | microbiological, Gram stain |
| 0 | Cell product for infusion (final | BM-MSC plus | cell number, viability, |
| | mixture of autologous and/or | BM-MNC | microbiological, Gram stain, |
| | allogeneic BM-MSC and | suspension | endotoxin |
| | autologous BM-MNC) | | |

BM-MNC: bone marrow-derived mononuclear cell fraction
BM-MSC: bone marrow-derived mesenchymal stem cells

In an example embodiment, cell infusion (transplantation) is completed in myocardial infarct patients intraoperatively in conjunction with coronary artery bypass grafting by direct injection following the circumference of the infarct border or via intracoronary percutaneous balloon catheter designed for angioplasty. Subjects may include patients who fit criteria for acute myocardial infarction or patients with a defined region of myocardial dysfunction related to a previous myocardial infarction.

In other example embodiments, cell infusion (transplantation) is completed in CD patients by injecting the cell mixture intracoronarily (by the use of a catheter), intracardially (directly into the heart) or intramusculary (in the arm, leg, etc in close proximity to an ischemic region without proper blood supply). Subjects may include patients who fit criteria for cardiovascular dysfunction associated with the existence of ischemic regions.

Improvement in function and tissue perfusion is evaluated by magnetic resonance imaging (“MRI”), echocardiography, angiography and 99mTc-TF perfusion scintigraphy.

Methods of CD replacement therapy for a patient are disclosed. The methods involve acquiring two types of bone marrow-derived cells—(1) a therapeutically effective amount of autologous or allogeneic mesenchymal stem cells that give rise to cardiomyocytes and/or endothelial repair cells and (2) a source of endothelial progenitor cells present as such, in the bone marrow-derived mononuclear cell fraction, that may give rise to new blood vessels. The therapeutically effective amount of mesenchymal stem cells and mononuclear cells are combined into an injection medium and the resulting mixture is injected into the patient.

This method may be used wherein the step of acquiring a therapeutically effective amount of autologous or allogeneic mesenchymal stem cells that give rise to cardiomyocytes and/or endothelial repair cells comprises performing a first bone marrow aspiration on the patient or other suitable human donor and producing a therapeutically effective amount of expanded bone marrow-derived mesenchymal stem cells, wherein the first bone marrow aspiration is performed at least twenty-five (25) days before the patient receives said injection medium. Alternatively, the first aspiration may be performed a sufficient amount of time before the injection, allowing enough time to expand the BM-MSCs until the therapeutically effective amount is reached.

Further, the present invention for myocardial and/or cardiovascular replacement therapy includes acquiring a source of a therapeutically effective amount of the autologous expanded bone marrow-derived mononuclear cells as a source of endothelial progenitor cells and comprises performing a second bone marrow aspiration from the patient's iliac crest on the day when the patient is to receive the injection medium, preferably, though not limited to, five hours before the patient is to receive the injection medium.

As another alternate, the second aspiration may be performed on the day when it is determined that the amount of mesenchymal stem cells is sufficient to produce the therapeutically effective amount of mesenchymal stem cells needed for the treatment, and not necessarily five hours prior to the infusion.

In another example though non-limiting embodiment, the method may be used not only for treatment of MI, but also for other CD, like: (1) vascular blood flow restoration; (2) increased blood flow supply due to vascular damage; and (3) capillary recruitment for severe vascular compromise.

While a number of example embodiments of the present invention have been described, it is understood that these example embodiments are illustrative only, and not restrictive, and that many modifications would be apparent to those of ordinary skill in the art.

### REFERENCES

Allers C, Sierralta W D, Neubauer S, Rivera F, Minguell J J, Conget P A. Dynamic of distribution of human bone marrow-derived mesenchymal stem cells after transplantation into adult unconditioned mice. Transplantation 78, 503, 2004
Assmus B, Schachinger V, Teupe C, Britten M, Lehmann R, Dobert N, Grunwald F, Aicher A, Urbich C, Martin H, Hoelzer D, Dimmeler S, Zeiher A M. Transplantation of Progenitor Cells and Regeneration Enhancement in Acute Myocardial Infarction (TOPCARE-AMI). Circulation 2002; 06: 3009-3017.
Barbash I M, Chouraqui P, Baron J et al. Systemic delivery of bone marrow-derived mesenchymal stem cells to the infarcted myocardium. Circulation. 2003; 108: 863.

Beltrami A P, Urbanek K, Kajstura J, Yan S M, Finato N, Bussani R, Nadal-Ginard B, Silvestri F, Leri A, Beltrami C A, Anversa P. Evidence that human cardiac myocytes divide after myocardial infarction. N Engl J. Med. 2001; 344:1750-1757.
Bittira B, Kuang J Q, Al-Khaldi A, Shum-Tim D, Chiu R C. In vitro pre-programming of marrow stromal cells for myocardial regeneration. Ann Thorac Surg. 2002; 74: 1154-1159.
Bittira B, Shum-Tim D, Al-Khaldi A, Chiu R C. Mobilization and homing of bone marrow stromal cells in myocardial infarction. Eur J Cardiothorac Surg. 2003; 24: 393-398.
Herzog E L, Chai L, Krause D S. Plasticity of marrow-derived stem cells. Blood 2003; 102: 3483-3493.
Husnain H K, Ashraf M. Bone marrow stem cell transplantation for cardiac repair. Am J Physiol Heart Circ Physiol 2005; 288: H2557-H2567.
Jackson K A, Majka S M, Wang H, Pocius J, Hartley C J, Majesky M W, Entman M L, Michael L H, Hirshi K K, Godell M A. Regeneration of ischemic cardiac muscle and vascular endothelium by adult stem cells. J Clin Invest 2001; 107: 1395-1402
Makino S, Fukuda K, Miyoshi S, Konishi F, et al. Cardiomyocytes can be generated from marrow stromal cells in vitro. J Clin Invest. 1999; 103: 697-705.
Minguell J J, Erices A, Conget P. Mesenchymal stem cells. Exp. Biol. Med. 2001; 226, 507-517.
Minguell J J, Erices, A. Mesenchymal Stem Cells and the Treatment of Cardiac Disease. Experimental Biology and Medicine, 2006; 231:39-49.
Min J Y, Sullivan M F, Yang Y, Zhang J P, Converso K L, Morgan J P, Xiao Y F.

Significant improvement of heart function by cotransplantation of human mesenchymal stem cells and fetal cardiomyocytes in postinfarcted pigs. Ann Thorac Surg. 2002, 74: 1568-1575.
Orlic D et al. Bone marrow cells regenerate infarted myocardium. Nature 2001; 410, 701-705.
Perin E C, Dohmann H F, Borojevic R, Silva S A, Sousa A L, et al. Transendocardial, autologous bone marrow cell transplantation for severe, chronic ischemic heart failure. Circulation. 2003; 107:2294-2302.
Pittenger M F, Martin B J. Mesenchymal stem cells and their potential as cardiac therapeutics. Circ Res. 2004; 95:9-20.
Rafii S, Lyden D. Therapeutic stem and progenitor cell transplantation for organ vascularization and regeneration. Nat. Med. 2003; 9: 702-712.
Sekiya, 2002 I, Larson B L, Smith J R, Pochampally R, Cui J G, Prockop D J. Expansion of human adult stem cells from bone marrow stroma: conditions that maximize the yields of early progenitors and evaluate their quality. Stem Cells, 2002; 20: 530-541.
Shake J G, Gruber P J, Baumgai luer W A, Senechal G, Meyers J, Redmond J M, Pittenger M F, Martin B J. Mesenchymal stem cell implantation in a swine myocardial infarct model: engraftment and functional effects. Ann Thorac Surg. 2002; 73: 1919-1925.
Siminiak T, Kurpisz M. Myocardial replacement therapy. Circulation 2003; 108:1167-1171 [0034] Stamm C, Westphal B, Kleine H D et al. Autologous bone-marrow stem-cell transplantation for myocardial regeneration. Lancet, 2003; 361: 45-46.
Strauer B E, Brehm M, Zeus T et al. Repair of infarcted myocardium by autologous intracoronary mononuclear bone marrow cell transplantation in humans. Circulation 2002; 106: 1913-1918.
Tse H F, Kwong Y L, Chan J K, Lo G, Ho C L, Lau C P. Angiogenesis in ischaemic myocardium by intramyocardial autologous bone marrow mononuclear cell implantation. Lancet. 2003; 361: 47-49.
Wagers A J, Christensen J L, Weissman I L. Cell fate determination from stem cells. Gene Therapy 2002; 9:606-612.
Wang J S, Shum-Tim D, Galipeau J, Chedrawy E, Eliopoulos N, Chiu R C. Marrow stromal cells for cellular cardiomyoplasty: feasibility and potential clinical advantages. J Thorac Cardiovasc Surg. 2000; 20: 999-1005.
Warejcka D J, Harvey R, Taylor B J, Young H E, Lucas P A. A population of cells isolated from rat heart capable of differentiating into several mesodermal phenotypes. J Surg Res 1996; 62:233-242.
Wulf G G, Jackson K A, Goodell M A. Somatic stem cell plasticity: current evidence and emerging concepts. Exp. Hematol. 2001; 29: 1361-1370.
Minguell J J, Florenzano F M, Ramirez M R, Martinez R F, Lasala G P. Intracoronary infusion of a combination of bone marrow-derived stem cells in dogs. Exp Clin Cardiol. 2010;15:17-20.
Lasala G P, Silva J A, Gardner P A, Minguell J J. Combination stem cell therapy for the treatment of severe limb ischemia: safety and efficacy analysis. Angiology. 2010;61:551-6.
Combination stem cell therapy for the treatment of medically refractory coronary ischemia: a Phase I study by Lasala G P, Silva J A, Kusnick B A, Minguell, J J. Cardiovascular Revascularization Medicine 2010, in press.

We claim:

1. A method for myocardial and/or cardiovascular replacement therapy comprising:
acquiring a therapeutically effective amount of mesenchymal stem cells, wherein said mesenchymal stem cells are autologous or allogeneic cells;
acquiring a therapeutically effective amount of mononuclear cells, wherein said mononuclear cells are autologous cells;
combining said therapeutically effective amount of mesenchymal stem cells and said therapeutically amount of mononuclear cells into an injection medium;
and injecting said injection medium into a patient in need of myocardial and/or cardiovascular replacement therapy.

2. The method for myocardial and/or cardiovascular replacement therapy of claim 1, wherein said mesenchymal stem cells are acquired through performing a first bone marrow aspiration on said patient or a suitable human donor to obtain a first aspirate; and
expanding and purifying said first aspirate until the therapeutically effective amount of mesenchymal stem cells is produced.

3. The method of myocardial and/or cardiovascular replacement therapy of claim 2, wherein said first bone marrow aspiration occurs at least twenty-five days before the patient is to receive said injection medium; and said first aspirate is aspirated from the patient's or the donor's iliac crest.

4. The method for myocardial and/or cardiovascular replacement therapy of claim 1, wherein said mononuclear cells are acquired through performing a second bone marrow aspiration on said patient to obtain a second aspirate; and analyzing said second aspirate to confirm that the therapeutically effective amount of mononuclear cells has been aspirated.

5. The method for myocardial and/or cardiovascular replacement therapy of claim 4, wherein said second bone marrow aspiration occurs on the same day said injection medium is to be injected into said patient; and said second aspirate is aspirated from the patient's iliac crest.

6. The method for myocardial and/or cardiovascular replacement therapy of claim 1, wherein injecting said injection medium is accomplished by intraoperatively injecting said injection medium directly to said patient's heart in conjunction with coronary artery bypass grafting or by any other transendocardial delivery system.

7. The method for myocardial and/or cardiovascular replacement therapy of claim 1, wherein injecting said injection medium is accomplished via an intracoronary catheter.

8. The method for myocardial and/or cardiovascular replacement therapy of claim 1, wherein injecting said injection medium is accomplished by injecting said injection medium intramuscularly into the patient.

9. The method for myocardial and/or cardiovascular replacement therapy of claim 1, wherein injecting said injection medium is accomplished by injecting said injection medium intravenously into the patient.

10. The method for myocardial and/or cardiovascular replacement therapy of claim 4, wherein said second aspiration occurs on a day when said therapeutically effective amount of autologous or allogeneic mesenchymal stem cells has been produced.
